(19) United States
(12) Patent Application Publication
(10) Pub. No.: US 2018/0161015 A1
(43) Pub. Date: Jun. 14, 2018

(54) VARIABLE SPEED OF SOUND BEAMFORMING BASED ON AUTOMATIC DETECTION OF TISSUE TYPE IN ULTRASOUND IMAGING

(71) Applicant: General Electric Company, Schenectady, NY (US)

(72) Inventors: Branislav Hollaender, Zipf (AT); Christian Perrey, Zipf (AT)

(21) Appl. No.: 15/374,420

(22) Filed: Dec. 9, 2016

(51) Int. Cl. A61B 8/00; A61B 8/08

(52) U.S. Cl. CPC A61B 8/54; A61B 8/462; A61B 8/5207; A61B 8/483; A61B 8/466

(57) ABSTRACT

Systems and methods are provided for variable speed of sound beamforming based on automatic detection of tissue type in ultrasound imaging.

### FIELD

Aspects of the present disclosure relate to medical imaging. More specifically, certain embodiments relate to methods and systems for variable speed of sound beamforming based on automatic detection of tissue type in ultrasound imaging.

### BACKGROUND

Various medical imaging techniques may be used, such as in imaging organs and soft tissues in a human body. Examples of medical imaging techniques include ultrasound imaging, computed tomography (CT) scans, magnetic resonance imaging (MRI), etc. The manner by which images are generated during medical imaging depends on the particular technique.

For example, ultrasound imaging uses real time, non-invasive high frequency sound waves to produce ultrasound images, typically of organs, tissues, objects (e.g., fetus) inside the human body. Images produced or generated during medical imaging may be two-dimensional (2D), three-dimensional (3D), and/or four-dimensional (4D) images (essentially real-time/continuous 3D images). During medical imaging, imaging datasets (including, e.g., volumetric imaging datasets during 3D/4D imaging) are acquired and used in generating and rendering corresponding images (e.g., via a display) in real-time.

Conventional systems and methods may, however, fail to account (or sufficiently and efficiently do so) for the different types of tissues in the areas being images, resulting in imaging operations that can be costly, inefficient, and/or ineffective.

Further limitations and disadvantages of conventional and traditional approaches will become apparent to one of skill in the art, through comparison of such systems with some aspects of the present disclosure, as set forth in the remainder of the present application with reference to the drawings.

### BRIEF SUMMARY

System and methods are provided for variable speed of sound beamforming based on automatic detection of tissue type in ultrasound imaging, substantially as shown in and/or described in connection with at least one of the figures, as set forth more completely in the claims.

These and other advantages, aspects and novel features of the present disclosure, as well as details of one or more illustrated example embodiments thereof, will be more fully understood from the following description and drawings.

### BRIEF DESCRIPTION OF SEVERAL VIEWS OF THE DRAWINGS

FIG. 1 is a block diagram illustrating an example medical imaging system that supports variable speed of sound beamforming based on automatic detection of tissue type in ultrasound imaging.

FIG. 2 is a block diagram illustrating an example ultrasound that supports variable speed of sound beamforming based on automatic detection of tissue type in ultrasound imaging.

FIG. 3 illustrates a flowchart of an example steps that may be performed for ultrasound imaging with variable speed of sound beamforming based on automatic detection of tissue type.

### DETAILED DESCRIPTION

Various implementations in accordance with the present disclosure may be directed to variable speed of sound beamforming based on automatic detection of tissue type in ultrasound imaging.

An example ultrasound system in accordance with the present disclosure may comprise a probe that is operable to transmit ultrasound signals and receive echo ultrasound signals; and processing circuitry that is operable to generate ultrasound dataset, corresponding to an ultrasound image, based on echo ultrasound sound signals captured via the probe; process the ultrasound dataset; detect, based on the processing of the ultrasound dataset, a type of tissue associated with each of one or more parts of the ultrasound image; determine for each detected type of tissue a corresponding local sound speed; and control transmission and/or reception of ultrasound signals during subsequent imaging operations based on determined local sound speeds, wherein the control comprises at least one of setting parameters or making adjustments to account for local sound speed for each of the one or more parts. The local sound speeds may be determined based on pre-programmed data defining for each of one or more different types of tissue a corresponding sound speed.

In an example implementation, the ultrasound system may be operable to identify the anatomical feature and determine the one or more imaging parameters or settings using a deep learning and/or neural network based model. The deep learning and/or neural network based model is pre-trained for recognizing one or more anatomical features. The deep learning and/or neural network based model is pre-trained for selecting, for each recognized anatomical feature, one or more imaging optimization parameters or settings. The deep learning and/or neural network based model is configured and/or updated based on feedback data from one or more users, the feedback data relating to recognizing and/or optimizing imaging for particular anatomical features. The deep learning and/or neural network based model and/or updates to the deep learning and/or neural network based model are imported into the ultrasound system.

In an example implementation, the processing circuitry may be operable to process the ultrasound dataset to assess one or more local features corresponding to one or more parts of the ultrasound image, and detect the corresponding type of tissue associated with each of the one or more parts of the ultrasound image, based on the one or more local features. The one or more local features may comprise at least one of speckle pattern, speckle size, speckle shape, maximal intensity, average intensity, contrast, and cross-correlation between adjacent pixels.

In an example implementation, the transmission and/or reception of ultrasound signals in the ultrasound system comprise utilizing beamforming, and controlling of transmission and/or reception of ultrasound signals comprises controlling of beamforming related parameters or functions to account for the local sound speed for each of the one or more parts. In some instances, the processing circuitry may be operable to, when controlling the beamforming related parameters or functions, determine and apply, for each of the one or more parts, a time delay based on the corresponding local sound speed.

In an example implementation, the processing circuitry may be operable to segment ultrasound images generated based on echo ultrasound signals captured via the probe, into regions with constant speed of sound. In some instances, the processing circuitry may be operable to determine refraction angles for a plurality of regions in the ultrasound images, resulting from the segmenting, and adjust beamforming related functions associated with the transmission and/or reception of ultrasound signals based on the determined refraction angles.

An example method in accordance with the present disclosure may comprise, in an ultrasound imaging device: generating ultrasound dataset, corresponding to an ultrasound image, based on captured echo ultrasound sound signals; processing the ultrasound dataset; detecting, based on the processing of the ultrasound dataset, a type of tissue associated with each of one or more parts of the ultrasound image; determining for each detected type of tissue a corresponding local sound speed; and controlling transmission and/or reception of ultrasound signals during subsequent imaging operations based on determined local sound speeds, wherein the control comprises at least one of setting parameters or making adjustments to account for local sound speed for each of the one or more parts. The local sound speeds may be determined based on pre-programmed data defining for each of one or more different types of tissue a corresponding sound speed.

In an example implementation, the method comprises processing the ultrasound dataset to assess one or more local features corresponding to one or more parts of the ultrasound image, and detecting the corresponding type of tissue associated with each of the one or more parts of the ultrasound image, based on the one or more local features. The one or more local features may comprise at least one of speckle pattern, speckle size, speckle shape, maximal intensity, average intensity, contrast, and cross-correlation between adjacent pixels.

In an example implementation, the transmission and/or reception of ultrasound signals comprise utilizing beamforming; and the controlling of transmission and/or reception of ultrasound signals comprises controlling of beamforming related parameters or functions to account for the local sound speed for each of the one or more parts. In some instances, the method comprises, when controlling the beamforming related parameters or functions, determining and applying, for each of the one or more parts, a time delay based on the corresponding local sound speed.

In an example implementation, the method comprises segmenting ultrasound images generated based on echo ultrasound signals captured via the probe, into regions with constant speed of sound. In some instances, the method further comprises determining refraction angles for a plurality of regions in the ultrasound images, resulting from the segmenting, and adjusting beamforming related functions associated with the transmission and/or reception of ultrasound signals based on the determined refraction angles.

An example non-transitory computer readable medium, in accordance with the present disclosure, may have stored thereon a computer program having at least one code section, the at least one code section being executable by a machine for causing the machine to perform one or more steps comprising: automatically identifying(e.g., without requiring any input by the user), during medical imaging based on a particular imaging technique, an anatomical feature in an area being imaged based on a deep learning and/or neural network based model; automatically determining (e.g., without requiring any input by the user), based on the identifying of the anatomical feature, and using the deep learning and/or neural network based model, one or more imaging parameters or settings for optimizing imaging quality for the identified anatomical feature; configuring operations and/or function relating to the medical imaging based on the determined one or more imaging parameters or settings; acquiring based on the configuration, medical imaging datasets corresponding to the area being imaged; and generating, based on processing on the medical imaging datasets, one or more medical images for rendering.

In an example implementation, the one or more steps performed in the machine may comprise processing the ultrasound dataset to assess one or more local features corresponding to one or more parts of the ultrasound image, and detecting the corresponding type of tissue associated with each of the one or more parts of the ultrasound image, based on the one or more local features. The one or more local features may comprise at least one of speckle pattern, speckle size, speckle shape, maximal intensity, average intensity, contrast, and cross-correlation between adjacent pixels.

In an example implementation, the transmission and/or reception of ultrasound signals comprise utilizing beamforming; and the controlling of transmission and/or reception of ultrasound signals comprises controlling of beamforming related parameters or functions to account for the local sound speed for each of the one or more parts. In some instances, the one or more steps performed in the machine may comprise, when controlling the beamforming related parameters or functions, determining and applying, for each of the one or more parts, a time delay based on the corresponding local sound speed.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an embodiment,” “one embodiment,” “a representative embodiment,” “an example embodiment,” “various embodiments,” “certain embodiments,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

In addition, as used herein, the phrase “pixel” also includes embodiments where the data is represented by a “voxel.” Thus, both the terms “pixel” and “voxel” may be used interchangeably throughout this document.

Also as used herein, the term “image” broadly refers to both viewable images and data representing a viewable image. However, many embodiments generate (or are configured to generate) at least one viewable image. Further, with respect to ultrasound imaging, as used herein the phrase “image” is used to refer to an ultrasound mode such as B-mode, CF-mode and/or sub-modes of CF such as TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations, such as single or multi-core: CPU, Graphics Board, DSP, FPGA, ASIC, or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams.” Also, forming of images may be performed using channel combinations that may originate from more than one transmit event (e.g., synthetic aperture techniques).

In various embodiments, imaging processing, including visualization enhancement, to form images may be performed, for example, in software, firmware, hardware, or a combination thereof.

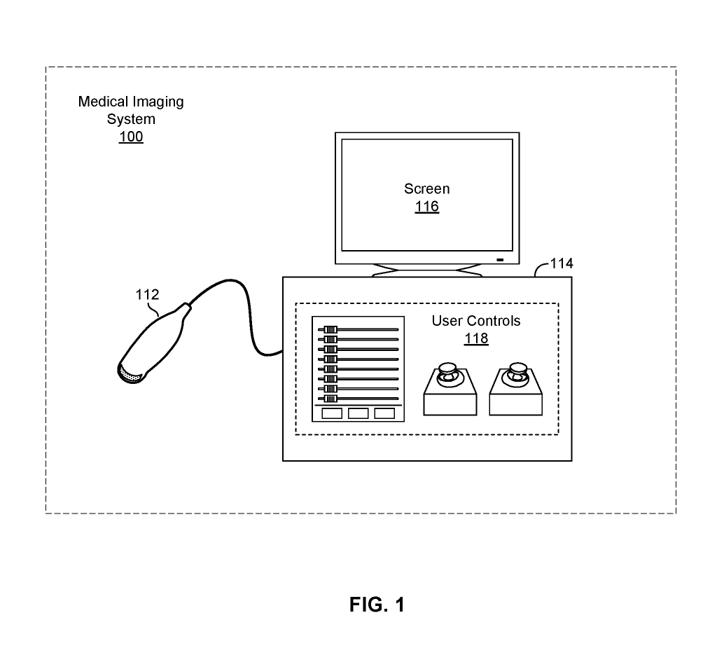

FIG. 1 is a block diagram illustrating an example medical imaging system that supports variable speed of sound beamforming based on automatic detection of tissue type in ultrasound imaging. Shown in FIG. 1 is an example medical imaging system 100.

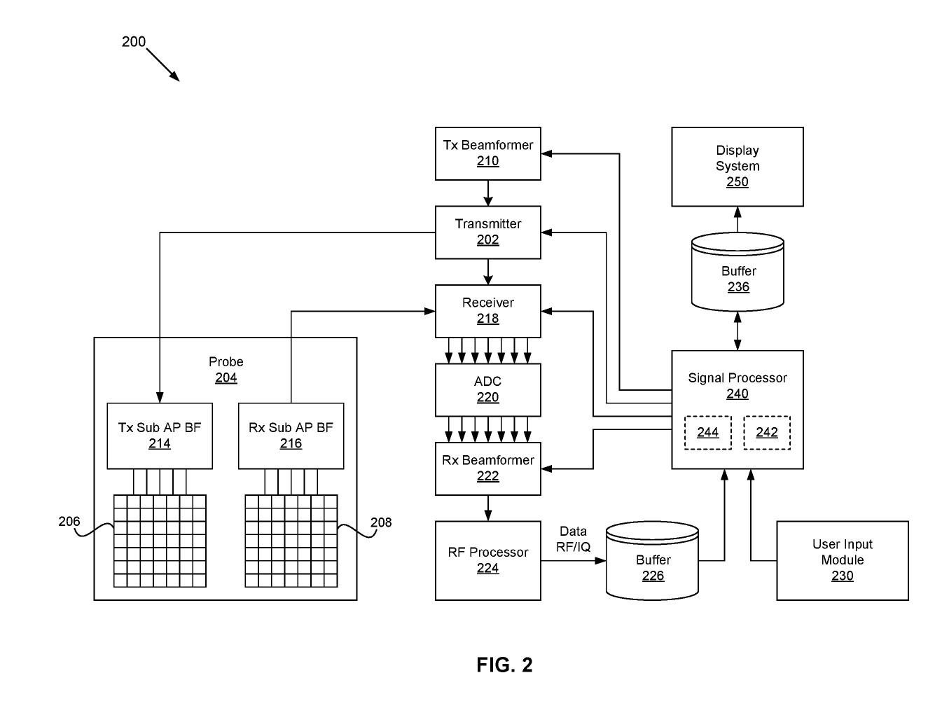

The medical imaging system 100 comprise suitable hardware, software, or a combination thereof, for supporting medical imaging—that is enabling obtaining data used in generating and/or rendering images during medical imaging exams. This may entail capturing of particular type of data, in particular manner, which may in turn be used in generating data for the images. For example, the medical imaging system 100 may be an ultrasound system, configured for generating and/or rendering ultrasound images. An example implementation of an ultrasound system that may correspond to the medical imaging system 100 is described in more detail with respect to FIG. 2.

As shown in FIG. 1, the medical imaging system 100 may comprise a probe 112, which may be portable and movable, and a display/control unit 114. The probe 112 may be used in generating and/or capturing particular type of signals (or data corresponding thereto), such as by being moved over a patient's body (or part thereof). For example, where the medical imaging system 100 is an ultrasound system, the probe 112 may emit ultrasound signals and capture echo ultrasound images.

The display/control unit 114 may be used in displaying images (e.g., via a screen 116). Further, the display/control unit 114 may also support user input/output. For example, the display/control unit 114 may provide (e.g., via the screen 116), in addition to the images, user feedback (e.g., information relating to the system, functions thereof, settings thereof, etc.). The display/control unit 114 may also support user input (e.g., via user controls 118), such as to allow controlling of the medical imaging. The user input may be directed to controlling display of images, selecting settings, specifying user preferences, requesting feedback, etc.

In operation, the medical imaging system 100 may be used in generating and presenting (e.g., rendering or displaying) images during medical exams, and/or in supporting user input/output in conjunction therewith. The images may be 2D, 3D, and/or 4D images. The particular operations or functions performed in the medical imaging system 100 to facilitate the generating and/or presenting of images depends on the type of system—that is the manner by which the data corresponding to the images is obtained and/or generated. For example, in ultrasound imaging, the data is based on emitted and echo ultrasound signals, as described in more detail with respect to FIG. 2.

In various implementations in accordance with the present disclosure, ultrasound imaging systems (such as, e.g., the medical imaging system 100, when implemented as ultrasound imaging system) may be configured to support and/or utilized variable speed of sound beamforming based on automatic detection of tissue type. In this regard, existing ultrasound systems typically utilize, and are configured to operate based on single and universal audio speed (e.g., 1540 m/s), irrespective of actual types of tissue in areas being imaged. However, sound may have different speed in different tissue types (e.g., muscle, fat, skin, connective tissue, etc.), and ultrasound imaging may be improved and optimized by using and/or accounting for such different sound speeds—that is, the actual local speed corresponding to each particular type of tissue. Accordingly, in various example implementations, local speeds of sound may be determined or estimated, and then utilized during ultrasound imaging.

For example, local speed of sound may be estimated based on the analysis of certain local properties of the image (e.g., speckle pattern, intensity, contrast, etc.) and subsequent recognition of tissue type (and therefore corresponding local speed of sound) based on these quantitative features. Local sound speeds may be pre-determined for various particular tissue types, and these pre-determined values may be stored into (or provided to) the system when needed/requested—e.g., when corresponding types of tissues are identified during active imaging.

FIG. 2 is a block diagram illustrating an example ultrasound that supports variable speed of sound beamforming based on automatic detection of tissue type in ultrasound imaging. Shown in FIG. 2 is an ultrasound system 200.

The ultrasound system 200 may comprise suitable components (physical devices, circuitry, etc.) for providing ultrasound imaging. The ultrasound system 200 may correspond to the medical imaging system 100 of FIG. 1 in ultrasound imaging use scenarios. The ultrasound system 200 comprises, for example, a transmitter 202, an ultrasound probe 204, a transmit beamformer 210, a receiver 218, a receive beamformer 222, a RF processor 224, a RF/IQ buffer 226, a user input module 230, a signal processor 240, an image buffer 236, and a display system 250.

The transmitter 202 may comprise suitable circuitry that may be operable to drive the ultrasound probe 204. The transmitter 202 and the ultrasound probe 204 may be implemented and/or configured for one-dimensional (1D), two-dimensional (2D), three-dimensional (3D), and/or four-dimensional (4D) ultrasound scanning. The ultrasound probe 204 may comprise a one-dimensional (1D, 2.25D, 2.5D or 2.75D) array or a two-dimensional (2D) array of piezoelectric elements. For example, as shown in FIG. 2, the ultrasound probe 204 may comprise a group of transmit transducer elements 206 and a group of receive transducer elements 208, that normally constitute the same elements. The transmitter 202 may be driven by the transmit beamformer 210.

The transmit beamformer 210 may comprise suitable circuitry that may be operable to control the transmitter 202 which, through a transmit sub-aperture beamformer 214, drives the group of transmit transducer elements 206 to emit ultrasonic transmit signals into a region of interest (e.g., human, animal, underground cavity, physical structure and the like). In this regard, the group of transmit transducer elements 206 can be activated to transmit ultrasonic signals. The ultrasonic signals may comprise, for example, pulse sequences that are fired repeatedly at a pulse repetition frequency (PRF), which may typically be in the kilohertz range. The pulse sequences may be focused at the same transmit focal position with the same transmit characteristics. A series of transmit firings focused at the same transmit focal position may be referred to as a “packet.”

The transmitted ultrasonic signals may be back-scattered from structures in the object of interest, like tissue, to produce echoes. The echoes are received by the receive transducer elements 208. The group of receive transducer elements 208 in the ultrasound probe 204 may be operable to convert the received echoes into analog signals, undergo sub-aperture beamforming by a receive sub-aperture beamformer 216 and are then communicated to the receiver 218.

The receiver 218 may comprise suitable circuitry that may be operable to receive and demodulate the signals from the probe transducer elements or receive sub-aperture beamformer 216. The demodulated analog signals may be communicated to one or more of the plurality of A/D converters (ADCs) 220.

Each plurality of A/D converters 220 may comprise suitable circuitry that may be operable to convert analog signals to corresponding digital signals. In this regard, the plurality of A/D converters 220 may be configured to convert demodulated analog signals from the receiver 218 to corresponding digital signals. The plurality of A/D converters 220 are disposed between the receiver 218 and the receive beamformer 222. Notwithstanding, the disclosure is not limited in this regard. Accordingly, in some embodiments, the plurality of A/D converters 220 may be integrated within the receiver 218.

The receive beamformer 222 may comprise suitable circuitry that may be operable to perform digital beamforming processing to, for example, sum the delayed channel signals received from the plurality of A/D converters 220 and output a beam summed signal. The resulting processed information may be converted back to corresponding RF signals. The corresponding output RF signals that are output from the receive beamformer 222 may be communicated to the RF processor 224. In accordance with some embodiments, the receiver 218, the plurality of A/D converters 220, and the beamformer 222 may be integrated into a single beamformer, which may be digital.

The RF processor 224 may comprise suitable circuitry that may be operable to demodulate the RF signals. In some instances, the RF processor 224 may comprise a complex demodulator (not shown) that is operable to demodulate the RF signals to form In-phase and quadrature (IQ) data pairs (e.g., B-mode data pairs) which may be representative of the corresponding echo signals. The RF (or IQ) signal data may then be communicated to an RF/IQ buffer 226.

The RF/IQ buffer 226 may comprise suitable circuitry that may be operable to provide temporary storage of output of the RF processor 224—e.g., the RF (or IQ) signal data, which is generated by the RF processor 224.

The user input module 230 may comprise suitable circuitry that may be operable to enable obtaining or providing input to the ultrasound system 200, for use in operations thereof. For example, the user input module 230 may be used to input patient data, surgical instrument data, scan parameters, settings, configuration parameters, change scan mode, and the like. In an example embodiment, the user input module 230 may be operable to configure, manage and/or control operation of one or more components and/or modules in the ultrasound system 200. In this regard, the user input module 230 may be operable to configure, manage and/or control operation of transmitter 202, the ultrasound probe 204, the transmit beamformer 210, the receiver 218, the receive beamformer 222, the RF processor 224, the RF/IQ buffer 226, the user input module 230, the signal processor 240, the image buffer 236, and/or the display system 250.

The signal processor 240 may comprise suitable circuitry that may be operable to process the ultrasound scan data (e.g., the RF and/or IQ signal data) and/or to generate corresponding ultrasound images, such as for presentation on the display system 250. The signal processor 240 is operable to perform one or more processing operations according to a plurality of selectable ultrasound modalities on the acquired ultrasound scan data. In some instances, the signal processor 240 may be operable to perform compounding, motion tracking, and/or speckle tracking. Acquired ultrasound scan data may be processed in real-time—e.g., during a B-mode scanning session, as the B-mode echo signals are received. Additionally or alternatively, the ultrasound scan data may be stored temporarily in the RF/IQ buffer 226 during a scanning session and processed in less than real-time in a live or off-line operation.

In operation, the ultrasound system 200 may be used in generating ultrasonic images, including two-dimensional (2D), three-dimensional (3D), and/or four-dimensional (4D) images. In this regard, the ultrasound system 200 may be operable to continuously acquire ultrasound scan data at a particular frame rate, which may be suitable for the imaging situation in question. For example, frame rates may range from 20-70 but may be lower or higher. The acquired ultrasound scan data may be displayed on the display system 250 at a display-rate that can be the same as the frame rate, or slower or faster. An image buffer 236 is included for storing processed frames of acquired ultrasound scan data that are not scheduled to be displayed immediately. Preferably, the image buffer 236 is of sufficient capacity to store at least several seconds' worth of frames of ultrasound scan data. The frames of ultrasound scan data are stored in a manner to facilitate retrieval thereof according to its order or time of acquisition. The image buffer 236 may be embodied as any known data storage medium.

In some instances, the ultrasound system 200 may be configured to support grayscale and color based operations. For example, the signal processor 240 may be operable to perform grayscale B-mode processing and/or color processing. The grayscale B-mode processing may comprise processing B-mode RF signal data or IQ data pairs. For example, the grayscale B-mode processing may enable forming an envelope of the beam-summed receive signal by computing the quantity (I²+Q²)^{1/2.} The envelope can undergo additional B-mode processing, such as logarithmic compression to form the display data. The display data may be converted to X-Y format for video display. The scan-converted frames can be mapped to grayscale for display. The B-mode frames that are provided to the image buffer 236 and/or the display system 250. The color processing may comprise processing color based RF signal data or IQ data pairs to form frames to overlay on B-mode frames that are provided to the image buffer 236 and/or the display system 250. The grayscale and/or color processing may be adaptively adjusted based on user input—e.g., a selection from the user input module 230, for example, for enhance of grayscale and/or color of particular area.

In some instances, ultrasound imaging may include generation and/or display of volumetric ultrasound images—that is where objects (e.g., organs, tissues, etc.) are displayed three-dimensional 3D. In this regard, with 3D (and similarly 4D) imaging, volumetric ultrasound datasets may be acquired, comprising voxels that correspond to the imaged objects. This may be done, e.g., by transmitting the sound waves at different angles rather than simply transmitting them in one direction (e.g., straight down), and then capture their reflections back. The returning echoes (of transmissions at different angles) are then captured, and processed (e.g., via the signal processor 240) to generate the corresponding volumetric datasets, which may in turn be used (e.g., via a 3D rendering module 242 in the signal processor 240) in creating and/or displaying volume (e.g. 3D) images, such as via the display 250. This may entail use of particular handling techniques to provide the desired 3D perception.

For example, volume rendering techniques may be used in displaying projections (e.g., 2D projections) of the volumetric (e.g., 3D) datasets. In this regard, rendering a 2D projection of a 3D dataset may comprise setting or defining a perception angle in space relative to the object being displayed, and then defining or computing necessary information (e.g., opacity and color) for every voxel in the dataset. This may be done, for example, using suitable transfer functions for defining RGBA (red, green, blue, and alpha) value for every voxel.

In various implementations in accordance with the present disclosure, the ultrasound system 200 may be configured to support variable speed of sound beamforming based on automatic detection of tissue type in ultrasound imaging. In particular, the ultrasound system 200 may be configured to assess the area being imaged to identify different types of tissue in it, and then perform ultrasound imaging based on actual local speeds of sound corresponding to each of the recognized types of tissue. In this regard, as noted above, sound may have different speed in different tissue types (e.g., muscle, fat, skin, connective tissue, etc.). Thus, quality of ultrasound images may be enhanced by using and/or accounting for the actual local speed corresponding to each particular type of tissue. In this regard, in ultrasound imaging, the image quality, in particular lateral resolution and contrast, is dependent on, at least in part, the transmit and receive beamforming process and data obtained based thereon.

Improving particular lateral resolution and contrast, and thus overall image quality, may be achieved based on knowledge (and use) of local sound speed in the imaged area. Existing systems and/or methods may be implemented in accordance with the incorrect assumption of a universal speed of sound in the human body, resulting in inferior image quality. In this regard, ultrasound beamforming processes in existing systems and methods are configured (e.g., use time delays adjusted based on) a single constant speed of sound, typically the universal sound speed of 1540 m/s. However, different tissues have varying speeds of sound due to their varying mechanical properties (e.g., 1450 m/s in fat, 1613 m/s in skin and connective tissue, etc.). The variations in speed of sound between the presumed universal sound speed and the actual local sound speed(s) may lead to incorrect focusing and/or increased clutter in generated images.

Thus, by knowing and using speed of sound accurately and locally in ultrasound imaging (e.g., the beamforming process) based on the actual local sound speeds for the tissue types in the imaged area, ultrasound image quality can be improved. For example, the transmit and receive beamforming process in the ultrasound system 200 may be configured to accommodate local variations in sound speed. Configuring ultrasound imaging (particularly, e.g., beamforming process used during such ultrasound imaging) in this manner would produce a perfectly focused image with higher contrast and resolution. Further, the geometry of the image may be rectified. This allows for more precise measurements. This may be particularly pertinent with particular types of patients (e.g., obese patients) and/or in exams of particular areas (e.g., breast imaging).

In an example implementation, an ultrasound system (e.g., the ultrasound system 200) may be configured to determine or estimate local speed of sound (e.g., via a sound speed control module 244 in the signal processor 240), such as based on an analysis of certain local properties and/or features (e.g., speckle pattern, speckle size and shape, intensity (including maximal and average intensity), contrast, cross-correlation between adjacent pixels and other higher-order statistical properties etc.) of an image obtained via ultrasound imaging, to recognize tissue types (and thus corresponding local speed of sound) from these quantitative features. These local speeds of sound may then be used in optimize the ultrasound imaging—e.g., in adjusting the time delay pattern in transmit and receive beamforming—that is, time delays applied to each of the received channel signals, which are summed to obtained the combined beamformed receive signal, thus improving the image quality. The sound speeds for various tissue types may be pre-stored into the system (e.g., within the signal processor 240, in a memory device (not shown), etc.), and accessed and used when needed—e.g., when corresponding types of tissues are identified during active imaging.

Detecting tissue types in this manner—that is, based on analysis of only local features rather than a full detection or segmentation of the acquired image/volume, is advantageous because of processing speed and simplicity of implementation (requiring very minimal, if any, changes to the already utilized hardware). For example, a standard delay-and-sum beamformer can be used with this technique. By adjusting the delay times of individual channels after the image analysis has been completed, the image can be enhanced. Further, data obtained based on analysis of local features can further be used for other purposes, such as detection and segmentation of organs or pathological defects.

In an example implementation, an ultrasound system (e.g., the ultrasound system 200) may be configured to perform (e.g., via the sound speed control module 244 of the signal processor 240) analysis of local image features, to identify the tissue type in a particular part of the image, by subdividing the image into an arbitrary number of parts, which are then analyzed individually, for determining the tissue type associated with each of the parts of the image. For example, a sliding window may be used to scan different portions in the image, to identify the tissue type associated with each portion. The tissue type may be determined or detected based on knowledge of local features associated with each of the different tissue types. Based on knowledge of sound speed in different tissue types, the local speed of sound can be estimated in every separate part of the image. The local features of the different tissues may be pre-programmed into the system. Alternatively, the system may be configured to determine (and store) these local features adaptively—e.g., in a separate learning process. For example, when imaging an already determined tissue type (e.g., based on user input, when performing a test image on known tissue type, etc.), the local features of the corresponding images may be assessed and stored for future use. The actual sound speeds associated with the different tissue types may be obtained in various ways. For example, the speed of sound for major tissue types in the human body may be well known, and as such may be pre-programmed into the systems. Further, in some instances, pre-programmed sound speeds may be tuned, such as based on actual use of the system.

In an example implementation, the adaptive adjustment of variable speed of sound beamforming based on automatic detection of tissue type may be configured as an iterative process. For example, in a first iteration, a universal speed of sound (e.g., 1540 m/s) may be used in the first iteration to construct an image using a known beamforming scheme. The local features of the beamformed image may then be analyzed, and time delays in the beamforming process may be adjusted according to the detected sound speeds. Using these adjusted time delays, an image may be obtained in a second iteration. This second image would presumably have a higher image quality. Optionally, more than two iterations can be used to further improve the image.

In an example implementation, detected local sound speeds may be used (e.g., via the signal processor 240) in segmenting images into regions with constant speed of sound. For example, by knowing the normals of region boundaries, refraction angles may be calculated. This data may then be incorporated into the beamforming process to further enhance the image.

In other example implementations, other techniques may be used for recognizing different types of tissue in areas being imaged and/or for adaptively adjusting ultrasound imaging operations to account for variation in local sound speed. For example, deterioration of image quality due to varying sound speeds in an imaged area may be addressed by omitting image analysis (e.g., including analysis of local features, as described above) and instead calculating correlation between radiofrequency (RF) signals of individual elements of the transducer. Time delays in the beamforming process may then be chosen so that these correlations are minimized. Such approach, however, requires that all element data be available to the processor. Further, this approach may require a change in the beamforming process and components used therefor. Further, a distinct feature in the image plane may be required to perform the computation, such as a point source. This may not be available in real-world imaging situations. Additionally, such approach usually assumes a single distorting layer between the tissue and the transducer (whereas with image analysis based approach, as described above, the speed of sound may be estimated in every analyzed window in the image). In another approach image analysis may be used, but with organ recognition being achieved based on machine learning techniques. In such approach knowledge about organ features (e.g., shape and texture) may be acquired, based on previously generated images, using learning algorithms, and that knowledge is then applied to new images for detection of organs (and thus type of tissue is determined from knowledge of tissue types associated with each organ). Such approach, however, requires more processing in comparison to the approach described above, which only requires analysis of local texture features and thus may be easier to implement, quicker, and less processing-intensive. In yet another approach, blind or non-blind deconvolution of an image may be used, using different kernels for different sound speeds. Such approach usually requires some way to automatically determine the image quality and to choose the best deconvolution kernel. This approach, however, may be slow and requires working globally and on the entire image.

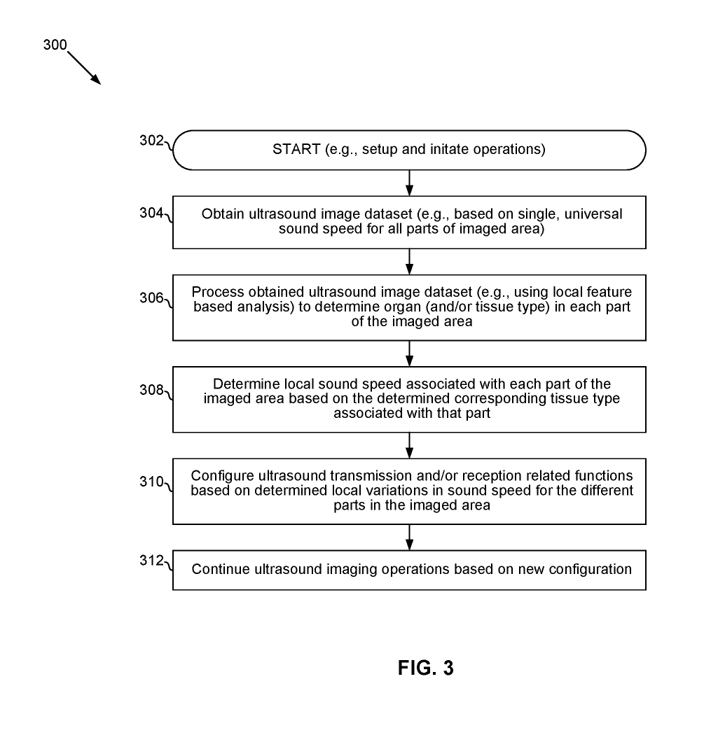

FIG. 3 illustrates a flowchart of an example steps that may be performed for ultrasound imaging with variable speed of sound beamforming based on automatic detection of tissue type. Shown in FIG. 3 is flow chart 300, comprising a plurality of example steps (represented as blocks 302-312), which may be performed in a suitable system (e.g., system 200 of FIG. 2) for performing ultrasound imaging with variable speed of sound beamforming based on automatic detection of tissue type.

FIG. 3 illustrates a flowchart of an example steps that may be performed for ultrasound imaging with variable speed of sound beamforming based on automatic detection of tissue type. Shown in FIG. 3 is flow chart 300, comprising a plurality of example steps (represented as blocks 302-312), which may be performed in a suitable system (e.g., system 200 of FIG. 2) for performing ultrasound imaging with variable speed of sound beamforming based on automatic detection of tissue type.

In start step 302, the system may be setup, and operations may initiate.

In step 304, ultrasound image dataset may be obtained (e.g., based on a single, universal sound speed, such as 1540 m/s, for all parts of imaged area).

In step 306, the obtained ultrasound image dataset may be processed (e.g., using image analysis based on local features, as described above) to determine corresponding organ and/or tissue type associated with each part of the imaged area.

In step 308, local sound speed associated with each part of the imaged area (i.e., local variations in speed of sound in the different parts of the imaged area) may be determined, based on the corresponding organ or tissue type associated with each part as determined in the previous step. The local sound speeds may be determined based on pre-programmed data defining the speed in particular tissue types.

In step 310, ultrasound transmission and/or reception related functions may be configured based on determined local variations in sound speed for the different parts in the imaged area. For example, for beamforming based operations, time delays may be calculated for each of the channel signals, with each time delay being determined based on local sound speed associated with the corresponding part in the imaged data.

In step 312, ultrasound imaging operations may be performed based on new configuration.

As utilized herein the terms “circuits” and “circuitry” refer to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.” As utilized herein, the terms “block” and “module” refer to functions than can be performed by one or more circuits. As utilized herein, the term “exemplary” means serving as a non-limiting example, instance, or illustration. As utilized herein, the terms “for example” and “e.g.,” set off lists of one or more non-limiting examples, instances, or illustrations. As utilized herein, circuitry is “operable” to perform a function whenever the circuitry comprises the necessary hardware (and code, if any is necessary) to perform the function, regardless of whether performance of the function is disabled or not enabled (e.g., by some user-configurable setting, a factory trim, etc.).

Other embodiments of the invention may provide a non-transitory computer readable medium and/or storage medium, and/or a non-transitory machine readable medium and/or storage medium, having stored thereon, a machine code and/or a computer program having at least one code section executable by a machine and/or a computer, thereby causing the machine and/or computer to perform the processes as described herein.

Accordingly, the present disclosure may be realized in hardware, software, or a combination of hardware and software. The present invention may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may be a general-purpose computing system with a program or other code that, when being loaded and executed, controls the computing system such that it carries out the methods described herein. Another typical implementation may comprise an application specific integrated circuit or chip.

Various embodiments in accordance with the present disclosure may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. An ultrasound system, comprising:
a probe that is operable to transmit ultrasound signals and receive echo ultrasound signals; and
processing circuitry that is operable to:
generate ultrasound dataset, corresponding to an ultrasound image, based on echo ultrasound sound signals captured via said probe;
process said ultrasound dataset;
detect, based on said processing of said ultrasound dataset, a type of tissue associated with each of one or more parts of said ultrasound image;
determine for each detected type of tissue a corresponding local sound speed; and
control transmission and/or reception of ultrasound signals during subsequent imaging operations based on determined local sound speeds, wherein said control comprises at least one of setting parameters or making adjustments to account for local sound speed for each of said one or more parts.

2. The ultrasound system of claim 1, wherein said processing circuitry is operable to:
process said ultrasound dataset to assess one or more local features corresponding to one or more parts of said ultrasound image; and
detect said corresponding type of tissue associated with each of said one or more parts of said ultrasound image, based on said one or more local features.

3. The ultrasound system of claim 3, wherein said one or more local features comprise at least one of speckle pattern, speckle size, speckle shape, maximal intensity, average intensity, contrast, and cross-correlation between adjacent pixels.

4. The ultrasound system of claim 1, wherein:
said transmission and/or reception of ultrasound signals comprise utilizing beamforming; and
said controlling of transmission and/or reception of ultrasound signals comprises controlling of beamforming related parameters or functions to account for said local sound speed for each of said one or more parts.

5. The ultrasound system of claim 4, wherein said processing circuitry is operable to, when controlling said beamforming related parameters or functions, determine and apply, for each of said one or more parts, a time delay based on said corresponding local sound speed.

6. The ultrasound system of claim 1, wherein said processing circuitry is operable to segment ultrasound images generated based on echo ultrasound signals captured via said probe, into regions with constant speed of sound.

7. The ultrasound system of claim 6, wherein said processing circuitry is operable to:
determine refraction angles for a plurality of regions in said ultrasound images, resulting from said segmenting; and
adjust beamforming related functions associated with said transmission and/or reception of ultrasound signals based on said determined refraction angles.

8. The ultrasound system of claim 1, wherein said processing circuitry is operable to determine local sound speeds based on pre-programmed data defining for each of one or more different types of tissue a corresponding sound speed.

9. A method, comprising:
in an ultrasound imaging device:
generating ultrasound dataset, corresponding to an ultrasound image, based on captured echo ultrasound sound signals;
processing said ultrasound dataset;
detecting, based on said processing of said ultrasound dataset, a type of tissue associated with each of one or more parts of said ultrasound image;
determining for each detected type of tissue a corresponding local sound speed; and
controlling transmission and/or reception of ultrasound signals during subsequent imaging operations based on determined local sound speeds, wherein said control comprises at least one of setting parameters or making adjustments to account for local sound speed for each of said one or more parts.

10. The method of claim 9, further comprising:
processing said ultrasound dataset to assess one or more local features corresponding to one or more parts of said ultrasound image; and
detecting said corresponding type of tissue associated with each of said one or more parts of said ultrasound image, based on said one or more local features.

11. The method of claim 10, wherein said one or more local features comprise at least one of speckle pattern, speckle size, speckle shape, maximal intensity, average intensity, contrast, and cross-correlation between adjacent pixels.

12. The method of claim 9, wherein:
said transmission and/or reception of ultrasound signals comprise utilizing beamforming; and
said controlling of transmission and/or reception of ultrasound signals comprises controlling of beamforming related parameters or functions to account for said local sound speed for each of said one or more parts.

13. The method of claim 12, further comprising, when controlling said beamforming related parameters or functions, determining and applying, for each of said one or more parts, a time delay based on said corresponding local sound speed.

14. The method of claim 9, further comprising segmenting ultrasound images generated based on echo ultrasound signals captured via said probe, into regions with constant speed of sound.

15. The method of claim 14, further comprising:
determining refraction angles for a plurality of regions in said ultrasound images, resulting from said segmenting; and
adjusting beamforming related functions associated with said transmission and/or reception of ultrasound signals based on said determined refraction angles.

16. The method of claim 9, further comprising determining local sound speeds based on pre-programmed data defining for each of one or more different types of tissue a corresponding sound speed.

17. A non-transitory computer readable medium having stored thereon, a computer program having at least one code section, said at least one code section being executable by a machine for causing said machine to perform one or more steps comprising:
generating ultrasound dataset, corresponding to an ultrasound image, based on captured echo ultrasound sound signals;
processing said ultrasound dataset;
detecting, based on said processing of said ultrasound dataset, a type of tissue associated with each of one or more parts of said ultrasound image;
determining for each detected type of tissue a corresponding local sound speed; and
controlling transmission and/or reception of ultrasound signals during subsequent imaging operations based on determined local sound speeds, wherein said control comprises at least one of setting parameters or making adjustments to account for local sound speed for each of said one or more parts.

18. The non-transitory computer readable medium of claim 17, the one or more steps further comprising:
processing said ultrasound dataset to assess one or more local features corresponding to one or more parts of said ultrasound image; and
detecting said corresponding type of tissue associated with each of said one or more parts of said ultrasound image, based on said one or more local features;

19. The non-transitory computer readable medium of claim 17, wherein:
said transmission and/or reception of ultrasound signals comprise utilizing beamforming; and
said controlling of transmission and/or reception of ultrasound signals comprises controlling of beamforming related parameters or functions to account for said local sound speed for each of said one or more parts.

20. The non-transitory computer readable medium of claim 19, the one or more steps further comprising, when controlling said beamforming related parameters or functions, determining and applying, for each of said one or more parts, a time delay based on said corresponding local sound speed.
